(19) United States
(12) Patent Application Publication
(10) Pub. No.: US 2013/0273637 A1
(43) Pub. Date: Oct. 17, 2013

(54) METHOD OF PROVISION OF SILICATE TO CULTURE MEDIA

(71) Applicants: Hywel David Griffiths, Auckland (NZ); Mark Humphrey Dines, Auckland (NZ)

(72) Inventors: Hywel David Griffiths, Auckland (NZ); Mark Humphrey Dines, Auckland (NZ)

(73) Assignee: PHOTONZ COPORATION LIMITED, Auckland (NZ)

(21) Appl. No.: 13/880,474

(22) PCT Filed: Oct. 21, 2011
(86) PCT No.: PCT/NZ2011/000222

§ 371 Date: Jun. 27, 2013

(30) Foreign Application Priority Data
Oct. 22, 2010 (NZ) 588781

(51) Int. Cl. C12N 1/12; C12M 1/00

(52) U.S. Cl. CPC C12N 1/12; C12M 29/00
USPC 435/257.1; 435/289.1

(57) ABSTRACT

The invention relates to a method for the provision of silicate to a culture media. In particular the invention relates to a method of mitigating silicate precipitation and/or gel formation when providing silicate to a culture media having a pH of about 7-9.

### FIELD OF INVENTION

The invention relates to a method for the provision of silicate to a culture media.

### BACKGROUND

Algae from the division Bacillariophyceae, commonly known as diatoms, are used and have the potential to be used for the production of a number of useful materials. These include use of whole algal biomass for aquaculture, use of metabolites of the algae, such as polyunsaturated fatty acids, for nutritional and pharmaceutical use, fatty acids for conversion to biofuel, amino acids for cosmetic use, pigments, and antibiotics (reviewed by Lebeau and Robert, Appl Microbial Biotechnol 60:612-623 2003). Diatoms are distinguished by the presence of a silica cell wall, and this too is useful in the formation of diatomaceous earth.

To form the silica cell wall, diatoms require the presence of bioavailable silicate or silicic acid in their growth environment. In the natural environment the silicate concentration is often low and whilst diatoms grow at relatively low densities under these circumstances this nutrient is still often limiting on their growth.

The utility of the diatoms and their products means that growth in a controlled environment is desirable in order to maximise productivity. High density culture in controlled environments is particularly advantageous to minimise capital costs of fermentation equipment, maximise productivity and ensure product consistency and security of supply. Controlled culture usually means that nutrients are added to a culture medium in which cells are grown. These nutrients include nitrogen sources, phosphate sources, vitamins, trace metals, and optionally an organic carbon source (for heterotrophic growth) or CO₂ (for photosynthetic growth). Controlled culture of diatoms also requires that silicate be added to the culture medium. This is usually done using sodium metasilicate, potassium metasilicate or sometimes silicic acid.

As has been noted by Suttle et al. (J. Phycol. 22:234-237 1986), difficulties are caused by formation of silicate polymers and precipitates during the preparation of silicate stocks and their use in the construction of culture media. This is due to the relationships between pH and solubility of silicates in that, when placed into culture media at a pH of less than around 10 (i.e. at a pH amenable to growth of most microalgae), silicate forms polymers or precipitates at concentrations above around 2 mM (depending on pH). The presence of salts in the culture medium may also exacerbate this problem. This has two consequences, one of which is to remove bio-available silicate from the diatom culture and the other is to foul the culture environment so as to interfere with the process, for example by occlusion of sensors. The precipitate may also remove other ions, such as trace metals, from solution, again negatively affecting the growth of the microalgae.

This is particularly a problem with high density culture where the silicate requirement of the culture over time may exceed the solubility limit, often by quite a considerable margin. One solution to this problem is to provide the silicate incrementally over the course of the culture growth. For example Kyle and Gladue in U.S. Pat. No. 5,244,921 and U.S. Pat. No. 5,567,732 indicate levels of between 5 and 7 g/L silicate are desirable over the course of their process and provide this in smaller amounts over the course of the fermentation. This method leads to a second problem in that, in order to minimise the effect of additional volume on the concentration of other media components, it is necessary to provide the metasilicate solution at concentrations measured in the region of grams per litre (for example in the region of 10-100 g/L). Alkali metal metasilicate salts are only soluble at these levels at high pH. As the metasilicate solution and culture medium come into contact, this leads to gradients in silicate concentration and pH along which precipitation occurs. This leads to the formation of silica deposits which can foul sensors, alter the mixing of cultures and can lead to blockage of valves, tubing and filters associated with the culture vessel.

Okauchi and Nakamura in US patent application 2009/0124501 release silicate into culture media slowly over time. They achieve this by producing a silica gel or sol that gradually dissociates to provide silicic acid to the culture solution. Whilst this may be suitable for use with slow growing, low density cultures it is unsuitable for use in situations where there is a high demand for silica. In addition, the technique is particularly unsuited to use in closed axenic fermentations where a sterile boundary needs to be maintained.

Kyle and Gladue in U.S. Pat. No. 5,244,921 and U.S. Pat. No. 5,567,732 note the problem of silicate precipitation if the concentration in media is allowed to reach too high a level. They state that “The undesired precipitation is avoided by feeding the metasilicate into the fermenter at a controlled rate.” However, the authors do not recognise that there is also a problem when the metasilicate stock comes into contact with culture media and do not suggest a means for overcoming this problem.

There is therefore a need in the art for methods by which concentrated silicate solutions may be added to culture media whereby the formation of silicate polymers (gels) or precipitates is avoided or at least mitigated.

### OBJECT OF THE INVENTION

It is an object of the invention to provide a method of mitigating silicate precipitation and/or gel formation when providing silicate to a culture media having a pH of about 7-9 which overcomes or ameliorates at least one of the disadvantages of the prior art.

It is a further alternative object to provide a method of growing microorganisms in culture media having a pH of about 7-9 which overcomes or ameliorates at least one of the disadvantages of the prior art.

It is a further alternative object to provide a culture vessel for growing microorganisms within a media.

It is a further alternative object to at least provide a useful choice to the public.

### SUMMARY OF THE INVENTION

According to a first aspect of the invention, there is provided a method of mitigating silicate precipitation and/or gel formation when providing silicate to a culture media having a pH of about 7-9 wherein the method comprises:
addition of an aqueous silicate solution to the culture media, wherein the aqueous silicate solution is rapidly dispersed into the culture media on addition to the culture media.

Preferably, the culture media is agitated to aid in the rapid dispersion of the aqueous silicate solution.

According to a second aspect of the invention, there is provided a method of mitigating silicate precipitation and/or gel formation when providing silicate to a culture media having a pH of about 7-9 wherein the method comprises:
addition of an aqueous silicate solution to the culture media,
agitating the culture media, and
wherein the first contact of the aqueous silicate solution with the culture media is at or proximate to an area of high or maximal agitation of the culture.

According to a third aspect of the invention, there is provided a method of growing microorganisms in culture media having a pH of about 7-9, the method including the steps of:
(i) agitating the culture media to substantially evenly distribute the microbial cells in the media;
(ii) adding aqueous silicate solution to the culture media at or proximate to an area of high or maximal agitation in the media.

Preferably, the culture media is agitated by stirring, shaking and/or by bubbles of gas passing through the culture media.

Preferably, the culture media is agitated to an extent such that the dissolved oxygen content of the culture is maintained above approximately 40%.

Preferably, the culture media comprises microalgae cells.

Preferably, the microalgae cells are diatoms.

Preferably, the silicate is an alkali metal silicate and/or silicic acid.

Preferably, the silicate is sodium metasilicate and/or potassium metasilicate.

Preferably, the silicate is sodium metasilicate.

Preferably, the silicate solution is added to the culture in a single application or multiple intermittent applications or continuous addition.

Preferably, the silicate solution is at a concentration prior to addition to the culture of at least about 50 mM, preferably at least about 100 mM, more preferably at least about 200 mM, even more preferably at least about 350 mM, even more preferably at least about 500 mM.

Preferably, the silicate solution is at a pH of greater than about 10 prior to addition to the culture, more preferably a pH of greater than about 12 prior to addition to the culture.

Preferably, the culture media is contained in a fermentation vessel.

Preferably, the culture media is maintained within a pH range of about 7-8.5, more preferably about 7.5-8.5.

Preferably, the pH of the culture media is controlled.

Preferably, the pH is controlled with acid and/or base.

Preferably, the silicate solution further comprises a basic component to aid in control of the pH of the culture medium.

Preferably, the basic component is sodium hydroxide and/or potassium hydroxide.

Preferably, the silicate solution is added to the culture media continuously.

Alternatively, the silicate solution is added to the culture media in a single application.

Alternatively, the silicate solution is added to the culture media in multiple intermittent applications.

Preferably, the silicate solution is added to the culture media via a conduit. In certain embodiments the conduit is a tube, pipe or nozzle.

Preferably, the conduit terminates above the surface of the culture media such that the silicate solution passes through a gaseous medium prior to contact with the culture media.

Preferably, the conduit terminates at a height above the surface of the culture media such the conduit is not in contact with any foam which may form on the surface of the culture media.

Preferably, the conduit terminates at a height above the surface of the culture media such that as the culture media is agitated or liquid is added to the culture media the conduit is not subject to splashes from the culture media surface.

Alternatively, the end of conduit from which the silicate is delivered to the culture media is shielded from splashes from the culture media surface.

Alternatively, the conduit terminates below the surface of the culture media.

Preferably, the linear velocity of the silicate solution in the conduit is at least 0.1 cm/sec as it enters the culture media, more preferably at least 0.5 cm/sec as it enters the culture media, more preferably at least 1 cm/sec as it enters the culture media more preferably at least 2 cm/sec as it enters the culture media, even more preferably at least 5 cm/sec as it enters the culture media.

Preferably, the linear velocity is selected to supply the needs of the microalgae.

Preferably, the internal diameter of the conduit is selected to give the required linear velocity of the silicate solution.

Preferably, the silicate solution is added to the culture media continuously.

Alternatively, the silicate solution is added to the culture media in a single application.

Alternatively, the silicate solution is added to the culture media in multiple intermittent applications.

Preferably, the conduit is purged with sterile gas or liquid following addition of silicate solution.

Preferably, the method further comprises providing the culture with aeration.

Preferably, the method further comprises providing the culture with light.

Preferably, the method further comprises providing further nutrients to the culture as the microalgal cells grow and use up the existing nutrients in the culture media.

Preferably, the method further comprises providing further silicate to the culture according to the method of the first or second aspect as the microalgal cells in the culture media grow and use up the existing silicate in the culture media.

Preferably, the method further comprises controlling the pH of the culture.

Preferably, the pH of the culture is controlled with acid and/or base.

Preferably, the pH of the culture is controlled by the addition of acid either separately to the culture or as part of the culture medium, in order to balance the effects of the silicate addition on pH.

For the avoidance of doubt, the above statements may be applied to the first, second or third aspects of the invention.

According to a fourth aspect of the invention, there is provided a process for producing microalgal biomass wherein the method comprises:
providing silicate to a culture comprising microalgal cells and a culture media according to the method of the first or second aspect;
harvesting biomass from the culture media.

According to a fifth aspect of the invention, there is provided a process for producing microalgal extracts wherein the method comprises:
providing silicate to a culture comprising microalgal cells and a culture media according to the method of the first or second aspect;
harvesting biomass from the culture media;
treating the biomass in such a manner as to separate one or more components from the biomass to form the microalgal extract.

Preferably, the process further comprises drying the biomass to reduce or eliminate water prior to treating.

Preferably, the process further comprises killing the cells to denature endogenous enzymes.

Preferably, the process further comprises forming the cells into a cake of biomass.

Preferably, treating the biomass comprises extracting the biomass with a non-selective or selective solvent and recovering the microalgal extracts from the solvent as a residue.

Preferably, the residue is further concentrated and/or purified.

According to a sixth aspect of the invention, there is provided a microalgal biomass produced by the method of the fourth aspect.

According to a seventh aspect of the invention, there is provided microalgal extracts produced by the method of the fifth aspect.

According to a eighth aspect of the invention, there is provided a culture vessel for growing microorganisms within a media, the vessel comprising,
a tank capable of containing culture media;
a conduit for delivering an aqueous silicate solution to the culture media;
   wherein the conduit is extendable or extends into the tank to terminate at either:
(i) an area above the surface of the culture media when contained in the tank; or
(ii) an area below the surface of the culture media when contained in the tank.

In certain embodiments the conduit is a tube, pipe or nozzle.

Preferably, the conduit terminates in (i) in an area to avoid contact of the silicate solution with tank component prior to contact with the culture media.

Preferably, the culture vessel further includes an agitation means associated with the tank, for agitating culture media contained in the tank.

Preferably, in the conduit is extendable or extends into the tank to terminate at either:
(i) an area above the surface of the culture media where there is high or maximal agitation of the culture media provided by the agitation means; or
(ii) an area below the surface of the culture media where there is high or maximal agitation of the culture media provided by the agitation means.

Preferably, the conduit terminates at a height above the surface of the culture media such that the conduit is not in contact with any foam which may form on the surface of the culture media.

Preferably, the conduit terminates at a height above the surface of the culture media such that as the culture media is agitated by the agitation means or solution is added to the culture media the conduit is not subject to splashes from the culture media surface.

Alternatively, the end of conduit from which the silicate is delivered to the culture media is shielded from splashes from the culture media surface.

Preferably, in use the linear velocity of the silicate solution in the conduit in (ii) is at least 0.1 cm/sec as it enters the culture media, more preferably at least 0.5 cm/sec as it enters the culture media, more preferably at least 1 cm/sec as it enters the culture media more preferably at least 2 cm/sec as it enters the culture media, even more preferably at least 5 cm/sec as it enters the culture media.

Preferably, the internal diameter of the conduit is selected to give the required linear velocity of the silicate solution.

Preferably, the vessel further includes a means to purge the conduit with sterile gas or liquid following addition of silicate solution.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

Embodiments of the invention will now be described, by way of example only, with reference to the accompanying drawings in which:

FIG. 1 shows a fermenter with tubing (A) installed to provide the silicate stock solution such that the stream of silicate stock solution drops through the atmosphere above the culture surface and falls into the culture into the region approximately above the mixing impellor (B).

FIG. 2 shows a fermenter with tubing (A) installed to provide the silicate stock solution such that the stream of silicate stock solution drops through the atmosphere above the culture surface and falls into the culture into the region approximately above the interior of the draught tube (B) where bubbles reach the surface and provide turbulent conditions.

FIG. 3 shows a fermenter with tubing (A) installed to provide the silicate stock solution such that the stream of silicate stock solution is delivered into the culture in the interior of the draught tube (B) below but proximate the surface where bubbles provide turbulent conditions.

FIG. 4 shows a top view of an unbaffled stirred tank fermenter indicating (with a star) an optimal position at which the silicate stock solution could be dropped through the atmosphere above the culture surface to fall into the culture to provide sufficient mixing for rapid dispersal.

FIG. 5 shows a top view of a baffled stirred tank fermenter with (a) a radial flow impellor or (b) a pitched blade or marine impellor indicating (with a star) an optimal position at which the silicate stock solution could be dropped through the atmosphere above the culture surface to fall into the culture to provide sufficient mixing for rapid dispersal. Note that these points are equidistant from the baffles. For the pitched blade or marine impellor the points are closer to the centre of the fermenter than for the radial flow impellor.

FIG. 6 shows a top view of an airlift fermenter with (a) airflow upwards through a concentric draft tube or (b) airflow upwards through the annulus. Optimal areas in which the silicate stock solution could be dropped through the atmosphere above the culture surface to fall into the culture to provide sufficient mixing for rapid dispersal are indicated with grey shading. For (b) note that the area adjacent to the fermenter wall is unsuitable.

### DEFINITIONS AND ABBREVIATIONS

Silicate: any of the ionised forms of monosilicic acid (Si(OH)₄) or metasilicic acid (H₂SiO₃).

Linear velocity: velocity along a line parallel to the bore of a conduit, for example a pipe, tube or nozzle.

CCAP: Culture Collection of Algae and Protozoa.

CCMP: The Provasoli-Guillard National Centre for Culture of Marine Phytoplankton.

UTEX: The Culture Collection of Algae at the University of Texas at Austin.

### DETAILED DESCRIPTION OF PREFERRED EMBODIMENTS

As has been previously noted herein, silicate is often required for growth of microorganisms, such as microalgae, in particular diatoms.

When the pH of a silicate solution drops below about 10, the solubility of the silicate also drops rapidly. The pH of a culture media must be amenable to the growth of microorganisms and therefore generally has a pH in the range of about 7 to about 9. At this pH the maximum solubility of a silicate solution can be as low as around between 1 mM and 2 mM (approximately 0.25 g/L and 0.5 g/L of sodium metasilicate pentahydrate) and precipitation/gel formation can occur.

There can arise, therefore, situations whereby growth of a microorganism requiring a silicate as a nutrient can exhaust the silicate which may be present in soluble form in culture media. It is therefore desirable to have means of providing more silicate to the culture media in the form of a silicate solution.

It is desirable for the silicate solution to be concentrated, for example in the region of about 50 mM to 500 mM (equivalent to about 10 to 100 g/L of sodium metasilicate pentahydrate), so that the additional volume added is not a significant proportion of the culture medium. This is particularly important for high density culture where the amounts of silicate required are high. Silicates, for example the alkali metal metasilicates (for example sodium metasilicate and potassium metasilicate) and/or silicic acid, are only soluble in water at such concentrations where the pH is greater than about 10. Therefore, the silicate solution is at a pH of greater than about 10 prior to addition to the culture, more preferably a pH of greater than about 12 prior to addition to the culture. The maximum pH of the silicate is about 14.

As noted above, culture media amenable to the growth of microorganisms is generally of pH lower than about 9, at which the solubility of silicate is low. There can arise, therefore, situations whereby addition of silicate solution at high concentration to culture media gives rise to localised mixtures with silicate concentration above around 2 mM (approximately 0.5 g/L of sodium metasilicate pentahydrate) and pH below about 10, where precipitation/gel formation occurs.

The inventors have carried out experiments where slow addition of a silicate solution to rapidly stirred culture media still results in undesired and unfavourable precipitation/gel formation of the silicate. The inventors have discovered that, where addition of the aqueous silicate solution is made such that the aqueous silicate solution is rapidly dispersed on addition to the culture media, the concentration of the silicate can be reduced sufficiently rapidly that precipitates/gels either do not form or their formation is reduced.

Reference to “rapidly dispersed” or the like, should be taken to mean that the concentration of silicate is reduced below the critical concentration of about 2 mM, more preferably below about 1 mM within a short time period. If, for example, a silicate stocks solution were used at a concentration of 500 mM then a 500-fold dilution within the time period is desired. Preferably the time period should be less than 1 second, more preferably less than 0.5 seconds, even more preferably less than 0.25 seconds, most preferably less than 0.1 seconds.

The inventors have identified that in order to mitigate (substantially reduce and/or stop) precipitate and/or gel formation of a silicate on addition of silicate solution to a culture media, the concentrated silicate solution should be rapidly dispersed through the culture media. The means by which silicate solution is added to the fermenter vessel has been found to be one method of achieving this. While not wishing to be bound by theory, the inventors believe if areas of concentrated silicate solution are allowed to remain, even for a short period of time, following addition of the silicate solution to the culture media, precipitates/gels will form. For example, the inventors have found when they allowed the silicate solution to gently run down the sides of the fermenter vessel, precipitate formed where the silicate solution contacted the culture medium, despite agitation being provided to the culture medium.

According to one embodiment of the invention, there is provided a method of mitigating silicate precipitation and/or gel formation when providing silicate to a culture media having a pH of about 7-9. The method comprises addition of an aqueous silicate solution to the culture media, wherein the concentration of the aqueous silicate solution reduces on addition to the culture media at a sufficient rate to prevent precipitation/gel formation of the silicate, i.e. there is rapid dispersion of the aqueous silicate solution on addition to the culture media.

Thus in a further embodiment the invention provides a method growing of microorganisms in a culture media, the method including the step of (i) adding an aqueous silicate solution to the culture media such that the silicate solution rapidly disperses in the culture media to a concentration of less than 2 mM to mitigate/prevent precipitation/gel formation of silicate in the culture media. More preferably the solution rapidly disperses in the culture media to a concentration of less than 1 mM to prevent precipitation/gel formation of silicate in the culture media. The rate of dispersal is preferably less than 1 second, more preferably less than 0.5 seconds, even more preferably less than 0.25 seconds, most preferably less than 0.1 seconds.

The culture media is preferably agitated to aid in the reduction of concentration of the aqueous silicate solution. Agitation can be provided, for example, by stirring of the culture media using impellors in the media, shaking of the culture media or vessel, and/or passing bubbles of gas through the culture media. Baffles within the culture vessel may also be used to aid in mixing. Preferably, the culture media is agitated to an extent such that the microbial cells within are substantially evenly distributed throughout the culture media. Preferably the agitation is sufficient to maintain the dissolved oxygen content of the culture media above approximately 40%.

One means of achieving the rapid dispersal of the silicate solution in the culture media is to add the aqueous silicate solution to the culture media such that the first contact of the added silicate solution is at an area of high agitation. Preferably the addition occurs at or proximate to, the area of highest, or maximum, agitation. Determination of such an area would be well within the knowledge of a person skilled in the art.

For example, when the culture media is in an airlift fermenter and air is provided by via a sparger at the base of a draught tube, the movement of the air drives circulation of the culture media up the centre of the draught tube. In this example, the silicate solution may be dropped into the culture media in the region directly above the draught tube, or via a conduit (for example a tube) which ends below the surface of the culture media within the interior of the draught tube, which will be the area of high or maximal agitation. In a further example, where the agitation is provided via stirring of the culture media, the silicate solution may be dropped into the culture media or fed into the culture below the surface, in the region above the mixing impellor, blade, propellers, paddles, and/or turbines which will be the area of high or maximal agitation.

Even distribution of cells in the media is very much preferred as this allows the added silicate solution to be distributed throughout the culture in an even manner. Thus growth across the media is maximized. Such matters are well within the knowledge of a person skilled in the art.

Thus the invention includes a method of growing microorganisms in culture media, the method including the steps of:
(i) agitating the culture media to substantially evenly distribute the microbial cells in the media;
(ii) adding aqueous silicate solution to the culture media such that the silicate solution rapidly disperses in the culture media.

Alternatively, the method includes the steps of:
(i) agitating the culture media to substantially evenly distribute the microbial cells in the media;
(ii) adding aqueous silicate solution to the culture media at or proximate to the area of highest agitation in the media.

According to a further embodiment of the invention, there is provided a method of at least mitigating silicate precipitation and/or gel formation when providing silicate to a culture media wherein the method comprises addition of an aqueous silicate solution to the culture media, wherein the culture media is agitated, and wherein the first contact of the aqueous silicate solution with the culture is at or proximate an area of high or maximal agitation of the culture.

The method of the invention may be used to add silicate to a culture media which does not contain silicate or to add further silicate to media which already contains silicate. The method may be used to add silicate to the culture media in a single application, multiple intermittent applications or as a continuous addition. The rate of addition is dependent on the requirements of the microalgae. Factors which affect this would be, for example, the cell density and growth rate. The rate of addition is also dependent on physical factors, for example the concentration of the silicate, volume of culture media, nature of the culture media (for example the pH and concentration of other salts in the media) and agitation rate. If the rate of addition of silicate solution to the media exceeds the rate at which it can be effectively diluted into the media precipitation/gel formation may occur. Once the desired set up of fermenter and culture are selected, a person skilled in the art would use standard experimental means to select an appropriate rate of addition of silicate solution. In one embodiment the rate is sufficient to maintain an average silicate concentration in the culture media in the range of about 250 μM to 1.25 mM (equivalent to about 50 mg/L to about 250 mg/L for sodium metasilicate pentahydrate), preferably in the range of about 0.5 mM to 1 mM (equivalent to about 100 mg/L to 200 mg/L for sodium metasilicate pentahydrate).

A culture media is a liquid or gel designed to support the growth of microorganisms or cells. Such a media comprises, for example, trace elements, salts, vitamins, bioavailable nitrogen sources (for example nitrates, yeast extracts and/or peptones) and optionally a carbon source (for example glucose or acetic acid).

In one embodiment, the culture media will also comprise microalgae. Collectively they may be referred to herein as a “culture” comprising microalgae and culture media. Reference is made herein to addition of silicate to a culture media. This should be taken to include embodiments in which silicate is to be added to a culture comprising microalgae and culture media. In one embodiment, the microalgae cells are diatoms. Diatoms have a silica cell wall and require the presence of bioavailable silicate in their growth environment. However, the method of the invention would be beneficial for addition of silicate to a culture media comprising any microorganism which requires or would benefit from the addition of silicate.

The silicate solution to be added to the culture media preferably comprises an aqueous solution of an alkali metal silicate and/or silicic acid, preferably an alkali metal metasilicate, preferably sodium metasilicate and/or potassium metasilicate, most preferably, the silicate is sodium metasilicate. The alkali metal metasilicates and silicic acid are a convenient source of silicate as they are soluble in water. Preferably, the concentration of the aqueous silicate solution (prior to addition to the culture) is at least about 50 millimolar (mM) (equivalent to about 10 g/L of sodium metasilicate pentahydrate), preferably at least about 100 mM (equivalent to about 20 g/L of sodium metasilicate pentahydrate) more preferably at least about 200 mM (equivalent to about 40 g/L of sodium metasilicate pentahydrate), even more preferably at least 500 mM (equivalent to about 100 g/L of sodium metasilicate pentahydrate). The concentration of silicate solution used will be dependent on the amount of silicate required. Where less silicate is required a lower concentration of solution may be used. Where there is a higher requirement of silicate, it may be preferable to use a higher concentration of silicate solution to minimise the effect of additional volume of solution on the concentration of other components in the culture media. The maximum concentration of the silicate solution will be dependent on the solubility of the silicate in aqueous solution, the temperature of the solution and the pH of the solution. The maximum concentration will be no more than 90% of the solubility maximum for the particular form of silicate at the operating temperature. For example, for sodium metasilicate pentahydrate at 30° C. this would be approximately 550 g/L or 2.58M.

Preferably, the silicate solution is at a pH of greater than about 10 prior to addition to the culture, preferably the pH is greater than about 12 prior to addition to the culture. Such levels of pH aid in the solubility of the silicate. The maximum pH of the silicate solution is about 14. Thus the pH range is preferably between about 10 and about 14.

The culture media is maintained at a pH of between about 7 and about 9, more preferably within a pH range of about 7-8, alternatively within a range of about 7-8.5, more preferably, within a range of about 7.5 to 8.5. To further aid in the control of the pH of the culture, a basic component may be added to the silicate solution, for example sodium hydroxide and/or potassium hydroxide. Such pH levels of the culture media are selected for being amenable to the growth of most microorganisms, more preferably microalgae, most preferably diatoms. However, the pH of the culture media may be selected according to the requirements of the microorganism used.

The silicate solution is preferably added to the culture media via a conduit, for example a tube, pipe or nozzle. Preferably, the conduit terminates above the surface of the culture media such that the silicate solution passes through the space above the culture media (gaseous medium) prior to contact with the culture media. Gaseous medium should be taken to mean any gas, but will preferably include oxygenated gas mixtures, preferably air. The conduit will extend or be extendable to protrude into the tank such that it terminates away from tank components such as the lid or walls or other solid surfaces (for example probes or baffles) of the tank in which the culture is contained. This achieves rapid disbursal of the silicate solution into the culture media. It is important that contact with the culture media is maximised and thus avoiding those tank components that are present (in particular the walls of the tank) is important when utilising this aspect.

Thus the invention includes a method of providing silicate to a culture media wherein the method comprises:
addition of an aqueous silicate solution to the culture media via a conduit which terminates above the surface of the culture media such that the silicate solution passes through a gaseous medium prior to contact with the culture media,
wherein the culture media is agitated, and
wherein the first contact of the aqueous silicate solution with the culture media is at or proximate an area of high or maximal agitation of the culture media.

Preferably, the conduit terminates at a height above the surface of the culture media such the conduit is not in contact with any foam which may form on the surface of the culture media. Preferably, the height of the conduit is such that the conduit is not subject to splashes from the culture media surface caused by, for example, mixing or addition of liquids to the culture medium. Alternatively, the end of conduit may be shielded from splashes from the culture media surface. For example, the end of the conduit could be shielded with a collar arrangement. Preferably the conduit terminates above a point or area on the culture surface that is highly turbulent, i.e. is an area of high or maximal agitation, to facilitate the rapid dispersal of the silicate stock solution into the culture media.

Alternatively, the conduit delivering the silicate solution can terminate below the surface of the culture media. If such an arrangement is used the inventors have found it is desirable to maintain certain flow rates of the silicate solution through the conduit in order to aid in the reduction of precipitate formation. Preferably, the linear velocity of the silicate solution is at least 0.1 cm/sec as it enters the culture, preferably at least 0.5 cm/sec, more preferably at least 1 cm/sec, even more preferably at least 2 cm/sec, most preferably at least 5 cm/sec. However, the selection of the flow rate will be dependent on the silicate requirement of the microorganisms (preferably microalgae, more preferably diatoms), and the concentration of the silicate solution. The maximum linear velocity of the silicate solution will be about 5 m/sec (thus a preferred range is between about 0.1 cm/sec and about 5 m/sec). While not wishing to be bound by theory, the inventors believe too low a flow rate results in gradients in silicate concentration and pH along which precipitation/gel formation can occur. To maintain a level of flow rate of the silicate solution in the conduit, preferably the silicate solution is added to the culture media as a continuous addition.

Thus the invention includes a method of providing silicate to a culture media wherein the method comprises:
addition of an aqueous silicate solution to the culture media via a conduit which terminates below the surface of the culture media such that the silicate solution,
wherein the culture media is agitated, and
wherein the first contact of the aqueous silicate solution with the culture media is at or proximate an area of high or maximal agitation of the culture media.

If the silicate solution is added to the culture media (preferably when contained in a fermentation vessel) via a conduit which terminates below the surface of the culture media and a single application or multiple intermittent applications are required, preferably the conduit is purged with sterile gas or liquid following the addition of the silicate solution. This may also be beneficial where the conduit terminates above the surface of the culture media, to maintain a clean conduit. However, this feature is more important where the conduit terminates below the surface of the culture media.

In a further embodiment of the invention, there is provided a method for the culture of microalgal cells wherein the method comprises providing silicate to a culture comprising microalgal cells and culture media according to the method of the first or second aspects.

Preferably, the method further comprises providing the culture with aeration and optionally providing light. Preferably the method further comprises providing further nutrients to the culture as the microalgal cells grow and use up the existing nutrients in the culture media.

Preferably, the method further comprises providing further silicate to the culture according to the method of the first or second aspects as the microalgal cells in the culture grow and use up the existing silicate in the culture media.

The method preferably further comprises controlling the pH of the culture with the addition of acid and/or base, in order to balance the effects of the silicate addition and cell growth on pH.

In a further embodiment of the invention, there is provided a process for producing microalgal biomass wherein the method comprises providing silicate to a culture comprising microalgal cells and culture media according to the method of the first or second aspects and harvesting the biomass from the culture media. Harvest may be achieved using standard methodology, for example flocculation, filtration or centrifugation of the culture.

The biomass may be further treated to separate one or more components from the biomass to form a microalgal extract. Therefore, in a further embodiment of the invention there is provided a process for producing microalgal extracts. For example, one method of treating the biomass may be to extract it with a non-selective or selective solvent and recover the microalgal extracts from the solvent as a residue. Such a residue may be further concentrated and/or purified as required. The process preferably further comprises drying the biomass to reduce or eliminate water prior to treating and killing the cells to denature endogenous enzymes.

A microalgal extract may contain one or more components of, and/or compounds produced by or contained in the microalgae. For example such components/compounds may include polyunsaturated fatty acids, fatty acids, amino acids, pigments, and antibiotics.

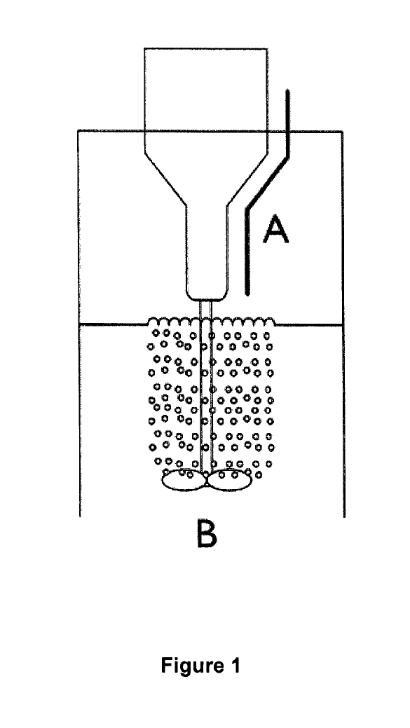

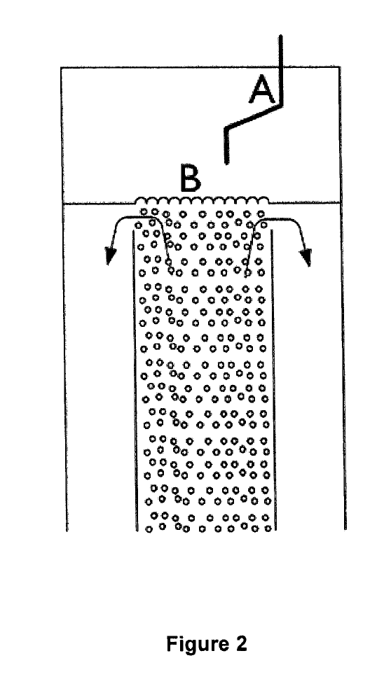

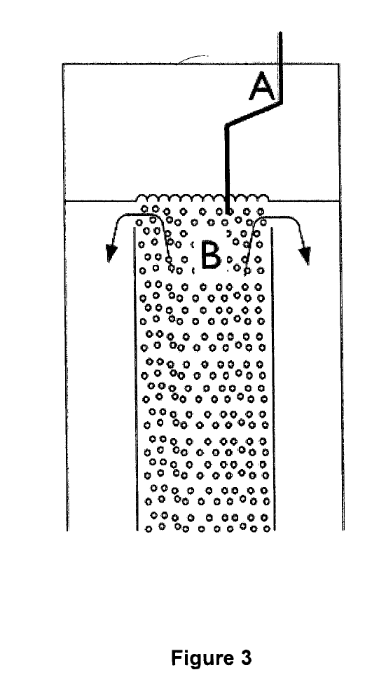

As seen best in FIGS. 1-3 a conduit (indicated by the bold line A) extends into and terminates within the culture media tank. This conduit allows addition of the aqueous silicate solution to the culture media (“B”) within the tank. In FIG. 1, the conduit A terminates above the surface of the culture media such that silicate solution will contact the culture at a point of high or maximal agitation, created by a stirring mechanism within media in the tank. As seen in FIG. 1, the high agitation area is at the surface of the media, in a central area away from the walls of the tank and away from other tank components (e.g. the stirring mechanism). FIG. 2 shows a similar device to FIG. 1 except that the culture media is agitated by using an airlift. Again this results in an area of high agitation at the surface of the culture media. The conduit (“A”) terminates above the area of high or maximal agitation and away from the walls of the tank. FIG. 3 shows an alternative option in which the conduit (“A”) terminates beneath the surface of the media. Again this is an area of high or maximal agitation (created by the airlift), and away from the walls of the tank.

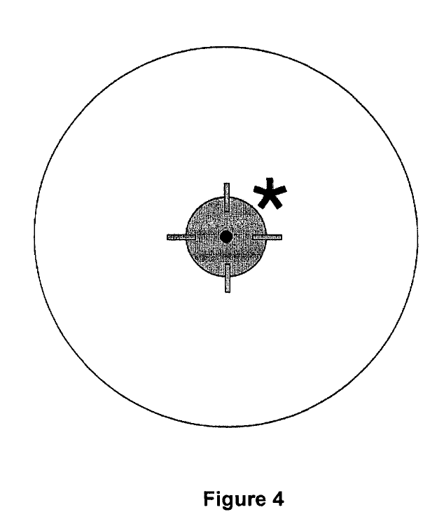

FIGS. 4 to 6 provide alternative indications of how the silicate solution could be added to culture media according to the invention. The silicate is added at points/areas of high or maximum agitation while avoiding as much as possible tank components (such as baffles, stirrers, walls etc).

FIG. 4 shows a top view of an unbaffled stirred tank fermenter indicating (with a star) an optimal position at which the silicate stock solution could be added. The solution would be dropped through the atmosphere in the tank above the culture surface to fall into the culture at an area to provide sufficient mixing for rapid dispersal.

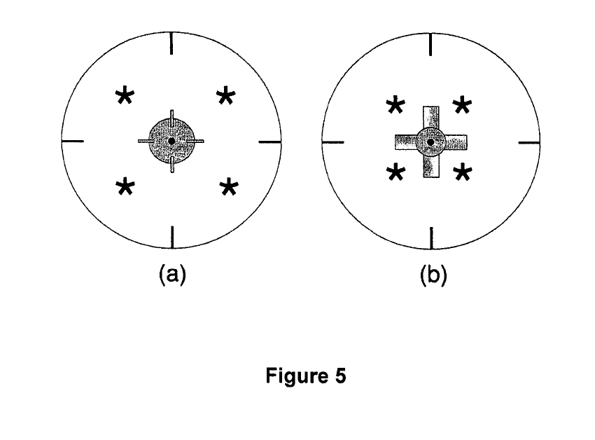

FIG. 5 shows a top view of a baffled stirred tank fermenter with (a) a radial flow impellor or (b) a pitched blade or marine impellor indicating (with a star) an optimal position (or positions) at which the silicate stock solution could be dropped through the atmosphere within the tank above the culture surface to fall into the culture to provide sufficient mixing for rapid dispersal. It can be noted that these points are equidistant from the baffles. The stars indicate the points of maximum or high agitation. For the pitched blade or marine impellor the points are closer to the centre of the fermenter than for the radial flow impellor.

In addition these points are chosen to minimise the potential for silicate solution to interact with the tank components (such as the baffles, tank walls, sensor, impellor shaft and the like) before the silicate solution contacts the culture media.

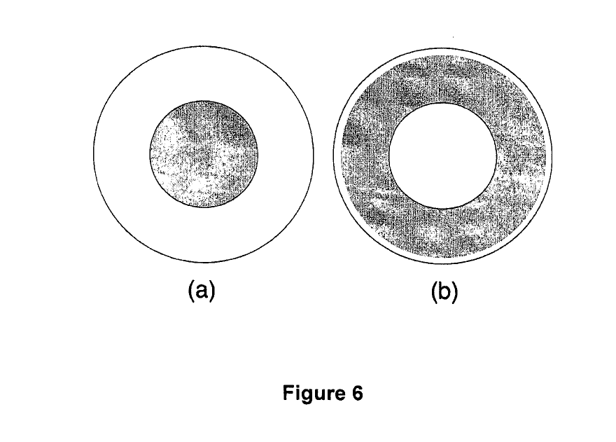

FIG. 6 shows a top view of an airlift fermenter with (a) airflow upwards through a concentric draft tube or (b) airflow upwards through the annulus. Optimal areas in which the silicate stock solution could be dropped through the atmosphere above the culture surface to fall into the culture to provide sufficient mixing for rapid dispersal are indicated with grey shading. For (a) the optimal area is centrally placed within the area of maximum agitation created. This is also away from the tank walls. For (b) it can be seen that the area adjacent to the fermenter (i.e. tank) wall is avoided while the optimal area is again within the area of maximal agitation created. As will be apparent, the further from the wall of the tank while remaining in the maximal agitation area is preferred.

Thus a further embodiment of the invention will be the apparatus used the in the method of the invention to provide silicate solution to a culture media. The apparatus will include a culture vessel for growing microorganisms within a media, the vessel comprising,
a tank capable of containing culture media;
a conduit for delivering an aqueous silicate solution to the culture media;
   wherein the conduit is extendable or extends into the tank to terminate at either:
(i) an area above the surface of the culture media when contained in the tank; or
(ii) an area below the surface of the culture media when contained in the tank.

The culture vessel can be constructed from commercially available components which would be readily available to a person skilled in the art. For example, the tank can be made from glass, steel or plastic, most preferably stainless steel. Fermentation tanks are commercially available from Applikon, New Brunswick, DiaChrom or the like. Alternatively they can be purposely built.

The conduit can be made can likewise be made of glass, steel or plastic, most preferably stainless steel and will be custom fitted to the tank. The conduit can be associated with the tank as it can be attached to the tank or be part of a separate device used specifically for addition reasons. As would be apparent, the agitation means can also be associated with the tank as it can be attached/integral with the tank (e.g. airlift) or separate from the tank (e.g. a shaking device on which the tank may sit or otherwise be connected).

### EXAMPLES

### Example 1

3 g (dry weight) of a Nitzschia laevis culture was transferred into each of two stirred tank fermenters with a working volume of 15 L. The vessels contained growth media with salts and vitamins together with nutrients including glucose, yeast extract, sodium nitrate, and potassium dihydrogen phosphate. Mixing was carried out using one Rushton and one marine impellor on a single shaft immersed in the culture and rotating at between 300 and 450 rpm.

The cultures were both aerated with one vessel volume of sterile air per minute and agitation controlled to give a dissolved oxygen content of >50%. pH was maintained at around 8.0 by the addition of 80 g/L sodium metasilicate. Temperature was maintained at 20° C. by the circulation of hot or cold water through a jacket around the fermenter vessels as required.

Above a certain cell density, harvest was carried out at 6 hourly intervals with harvested volume being replaced with fresh media to make a continuous culture.

Once harvest had commenced, sodium metasilicate pentahydrate solution (80 g/L w/v, approximately 380 mM) at a pH of around 12.5 was fed into the vessels in regularly spaced 0.05 mL volumes such that over time sufficient sodium metasilicate was provided to support the growth of the cells.

In fermenter 1 silicate solution was provided via a tube that ended flush with the top plate of the fermenter. In fermenter 2 (as is indicated in FIG. 1), tubing (A) was installed such that the incoming stream of metasilicate stock dropped through the air above the culture surface and fell into the culture into the region directly above the mixing impellor (B).

Fermenters were opened a week after the silicate feed had commenced and examined for the build up of precipitate.

In fermenter 1 it was apparent that, on entry into the vessel, sodium metasilicate solution had not dropped into the culture media but had run across the top plate of the fermenter and down baffles and sensors into the culture medium. Dilution of the silicate at the surface of the culture had not been rapid resulting in precipitate formation, which slowed the mixing further due to hold-up of solution in the precipitate. Significant build-up of precipitates was seen at the level of the culture surface on baffles and sensors. In one case this had begun to obstruct the sensor through fouling of a float.

In fermenter 2 no such build up of precipitate was seen either at the exit of the metasilicate stock line or at the level of the culture surface. It is believed that the 0.05 mL drops of metasilicate solution were mixed in the top litre of culture in the vessel in less than a second; a dilution factor of 20,000 fold per second or 2,000 fold per 0.1 second. The desired dilution factor is 380 mM to 1 mM.

Equal amounts of agitation were provided to both tanks and equal volume and concentration of silicate solution were added to both tanks. The precipitation of silicates was apparent in fermenter 1 in contrast to no build up of silicate precipitate in fermenter 2.

### Example 2

10 L of a 10 g/L culture (dry cell weight) of a Nitzschia laevis was transferred into an airlift fermenter with a working volume of 500 L. The vessel contained growth media with salts and vitamins together with nutrients including glucose, yeast extract, sodium nitrate, and potassium dihydrogen phosphate.

The airlift fermenter is an upright column with an internal concentric draught tube. Air is supplied via a sparger at the base of the draught tube and rises to the culture surface. This movement of air drives circulation of the culture medium up the centre of the draught tube and back down between the fermenter wall and draught tube.

The culture was aerated with sterile air controlled to give a dissolved oxygen content of >50% and to provide mixing. An airflow of around 100 L/min was found to be sufficient to provide this. pH was maintained at around 8.0 by the addition of 80 g/L (w/w) sodium metasilicate pentahydrate solution. Temperature was maintained at 20° C. by the continuous circulation of water at this temperature through a jacket around the fermenter vessel.

Once a suitable culture dry weight had been reached, harvest was carried out at 6 hourly intervals. Harvested volume was replaced with fresh media leading to a continuous culture with a stable cell density.

Once harvest had commenced, sodium metasilicate pentahydrate solution (80 g/L w/v, approximately 380 mM) at a pH of around 12.5 was fed into the vessels in regularly spaced 3 mL volumes such that over time sufficient sodium metasilicate was provided to support the growth of the cells.

Under these conditions we estimate that there is a flow of at least around 25 L per second through the draft tube of the fermenter. Dilution of the silicate solution is therefore around 3 ml into 25 L per second, more than 8 thousand fold in a second, 800-fold in 0.1 second. The desired dilution is 380 mM to 1 mM, 380-fold.

As is indicated in FIG. 2, tubing (A) was installed such that the incoming stream of metasilicate stock dropped through the air above the culture surface and fell into the culture into the region directly above the interior of the draught tube (B) where bubbles reach the surface and provide turbulent conditions.

After a period of operation of a week no build up of precipitate around the input tubing was detected.

### Example 3

10 L of a 10 g/L culture (dry cell weight) of a Nitzschia laevis is transferred into an airlift fermenter with a working volume of 500 L. The vessel contained growth media with salts and vitamins together with nutrients including glucose, yeast extract, sodium nitrate, potassium dihydrogen phosphate and 250 mg/L sodium metasilicate.

The culture is aerated with sterile air controlled to give a dissolved oxygen content of >50% and to provide mixing. An airflow of around 100 L/min is found to be sufficient to provide this. pH was maintained by the addition of 1N sodium hydroxide. Temperature is maintained at 20° C. by the continuous circulation of water at this temperature through a jacket around the fermenter vessel.

Once a suitable culture dry weight has been reached, harvest is carried out at 6 hourly intervals. Harvested volume is replaced with fresh media leading to a continuous culture with a stable cell density.

Once culture dry weight reaches around 3 g/L, sodium metasilicate pentahydrate solution (80 g/L w/v, approximately 380 mM) at a pH of around 12.5 is fed into the vessels in a continuous stream at a flow rate of 2.5 mL/min. Once harvest commences, flow rates are adjusted to between about 1.5 mL/min and 3.5 mL/min such that over time sufficient sodium metasilicate is provided to support the growth of the cells.

Metasilicate stock solution is delivered via stainless steel tubing with an external diameter of 3.2 mm (⅛ inch) and a wall thickness of 0.7 mm (0.028 inch) (Swagelok). At a flow rate of 2.5 mL/min the stock solution passes through this tubing at approximately 1.6 cm/sec. At a flow rate of 1.5 mL/min the stock solution passes through this tubing at approximately 1.0 cm/sec. 3.5 mL/min the stock solution passes through this tubing at approximately 2.3 cm/sec.

As is indicated in FIG. 3, tubing (A) is installed such that the incoming stream of metasilicate stock is delivered into the culture in the interior of the draught tube (B) below but close to the surface where bubbles provide turbulent conditions.

After a period of operation of a week no build up of precipitate around the input tubing is detected nor is any blockage of the input tubing experienced.

### Example 4

Areas of the culture surface have been considered in terms of the level of turbulence presence and the potential for rapid dispersal of silicate stock dropped in via the gaseous medium above the culture surface.

The position of the area at the culture surface with maximal mixing is going to be highly dependent upon the geometry of the fermenter and the method of mixing.

In a simple unbaffled stirred tank fermenter (FIG. 3) the optimal position may be into the vortex formed around the centre of the culture, although far enough from the shaft of any agitation device to avoid contact of the stock solution with that shaft.

In a baffled stirred tank fermenter (FIG. 4), the optimal position may be midway between baffles where opposing flows created by the baffles come together. With a pitched blade or marine impellor a vortex may also be formed bringing the optimal mixing position closer towards the centre of the culture.

In an airlift fermenter (FIG. 5), the optimal position could be anywhere in the area where bubbles arrive at the surface of the culture, i.e. above the section of the fermenter where there is an upward rather than downward flow of media.

The presence of sensors or other intrusions into the surface of the culture will affect the locations of turbulence at the culture surface but will generally increase the level of mixing. Care should, however, be taken to avoid the introduced concentrated silicate solution from coming into contact with any solid surface, such as probes, baffles or the fermenter wall, before it contacts the culture medium.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Wherein the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the scope of the invention.

1-39. (canceled)

40. A method of mitigating silicate precipitation and/or gel formation when providing silicate to a culture media having a pH of about 7-9 wherein the method comprises:
addition of an aqueous silicate solution to the culture media, wherein the aqueous silicate solution is rapidly dispersed into the culture media on addition to the culture media.

41. The method of claim 40 wherein the culture media is agitated to aid in the rapid dispersion of the aqueous silicate solution.

42. A method of mitigating silicate precipitation and/or gel formation when providing silicate to a culture media having a pH of about 7-9 wherein the method comprises:
addition of an aqueous silicate solution to the culture media,
wherein the culture media is agitated, and
wherein the first contact of the aqueous silicate solution with the culture media is at or proximate an area of high or maximal agitation of the culture.

43. The method of claim 42 wherein the culture media is agitated by stirring, shaking and/or by bubbles of gas passing through the culture media.

44. The method of claim 42 wherein the culture media comprises microalgae cells.

45. The method of claim 42 wherein the silicate is an alkali metal silicate and/or silicic acid.

46. The method of claim 42 wherein the silicate solution is at a concentration prior to addition to the culture of at least about 50 mM.

47. The method of claim 42 wherein the culture media is maintained at a pH of less than about 9.

48. The method of claim 42 wherein the silicate solution is added to the culture media continuously.

49. The method of claim 42 wherein the silicate solution is added to the culture media in a single application.

50. The method of claim 42 wherein the silicate solution is added to the culture media in multiple intermittent applications.

51. The method of claim 42 wherein the silicate solution is added to the culture media via a conduit, wherein the conduit terminates above the surface of the culture media such that the silicate solution passes through a gaseous medium avoiding contact of the silicate solution with tank components prior to contact with the culture media.

52. The method of claim 42 wherein the silicate solution is added to the culture media via a conduit, wherein the conduit terminates below the surface of the culture media.

53. The method of claim 52 wherein the linear velocity of the silicate solution in the conduit is at least 0.1 cm/sec as it enters the culture media.

54. A method of growing microorganisms in culture media having a pH of about 7-9, the method including the steps of:
(i) agitating the culture media to substantially evenly distribute the microbial cells in the media;
(ii) adding aqueous silicate solution to the culture media at or proximate to an area of high or maximal agitation in the culture media.

55. A culture vessel for growing microorganisms within a media, the vessel comprising,
a tank capable of containing culture media;
a conduit for delivering an aqueous silicate solution to the culture media;
wherein the conduit is extendable or extends into the tank to terminate at either:
(i) an area above the surface of the culture media when contained in the tank wherein the area avoids contact of the silicate solution with tank components prior to contact with the culture media; or
(ii) an area below the surface of the culture media when contained in the tank.

56. The culture vessel for growing microorganisms within a media of claim 55 wherein the culture vessel further includes an agitation means associated with the tank, for agitating culture media contained in the tank.

57. The culture vessel for growing microorganisms within a media of claim 56 wherein the area of (i) is above the surface of the culture media where there is high or maximal agitation of the culture media provided by the agitation means.

58. The culture vessel for growing microorganisms within a media of claim 57 wherein the conduit terminates at a height above the surface of the culture media such that the conduit is not in contact with any foam which may form on the surface of the culture media.

59. The culture vessel for growing microorganisms within a media of claim 55 wherein the conduit terminates at a height above the surface of the culture media such that as the culture media is agitated by the agitation means or solution is added to the culture media the conduit is not subject to splashes from the culture media surface.
